(19) United States
(12) Patent Application Publication
(10) Pub. No.: US 2017/0368528 A1
(43) Pub. Date: Dec. 28, 2017

(54) METHOD FOR MAKING A GAS FROM WATER, PRODUCT OF THE METHOD, AND APPARATUS THEREFOR

(71) Applicants: Robinson B. Gourley, Sarasota, FL (US); Ted Suratt, Cleanwater, FL (US)

(72) Inventors: Robinson B. Gourley, Sarasota, FL (US); Ted Suratt, Cleanwater, FL (US)

(21) Appl. No.: 15/697,623

(22) Filed: Sep. 7, 2017

Related U.S. Application Data
(63) Continuation-in-part of application No. 15/059,337, filed on Mar. 3, 2016.
Application No. 15/059,337 is a continuation-in-part of application No. 12/596,077, filed on Mar. 17, 2010, now abandoned.
Application No. 12/596,077 is a continuation-in-part of application No. 11/738,476, filed on Apr. 21, 2007, now abandoned.

(51) Int. Cl. B01J 19/08; A61K 9/08; A61K 31/375; C01B 5/00; C05D 9/00; F23J 7/00; A61K 8/19; A61Q 19/00; A61Q 11/00; A61K 9/00; A61K 33/00

(52) U.S. Cl. CPC B01J 19/087; A61K 9/0048; A61K 9/08; A61K 31/375; C01B 5/00; A61K 33/00; F23J 7/00; A61K 8/19; A61Q 19/00; A61Q 11/00; C05D 9/00; B01J 2219/0862; B01J 2219/0877; C01P 2006/60; B01J 2219/0854

(57) ABSTRACT

A method for producing a purified, stable, dioxytetrahydride compressible gas from water. The gas is suitable for a variety of uses and may also be infused into water which itself is useful for a variety of purposes.

### CROSS REFERENCE TO RELATED APPLICATIONS

This is a continuation-in-part of U.S. patent application Ser. No. 15/059,337, filed Mar. 3, 2016, currently pending, which is a continuation-in-part application of U.S. patent application Ser. No. 12/596,077, filed Mar. 17, 2010, abandoned, which is a 371 of PCT/US08/60666, filed Apr. 17, 2008, which is a Continuation-in-Part application of U.S. patent application Ser. No. 11/738,476, filed Apr. 21, 2007, from which priority is claimed.

### TECHNICAL FIELD

This invention relates to the generation of a purified stable gas from water, wherein said gas may be stored under relatively high pressure and can be used to infuse water.

### BACKGROUND OF THE INVENTION

The invention involves the use of strong electromagnetic fields using iron plates to transfer electrons through water-based electrochemical solutions. This electro-magnetic process binds two oxygens into a diatomic bond with four hydrogen atoms while adding electrons to hydrogen causing both stabilization of the liquid-gas structure and repulsion of other water molecules thereby creating a stable gaseous form of water that is dioxytetrahydride (i.e. H₄O₂). Dioxytetrahydride is an electron-rich gaseous form of water not created by electrolysis. The gas is created when very strong magnetic fields are applied to liquid water mixed in an electrochemical solution. Using an electromagnetic field of the instant invention, oxygen of two water molecules is brought into a diatomic bond. The four hydrogens pick up added electrons changing the bond angle between hydrogen and oxygen from 104° to 118° and thus creating a liquid type crystal structure, dioxytetrahydride. The addition of electrons prevents water molecules from forming a cluster type structure. In other words, the electrons repel other water molecules and this in turn, creates a gaseous form of water, dioxytetrahydride. It is the addition of electrons that repel water molecules to create the gas and not the breaking of either the hydrogen or oxygen bonds. This explains how relatively low energy below that used in normal electrolysis is used to convert liquid into gas.

Electrolysis of water is known to produce hydrogen gas (H₂) at a cathode and oxygen gas (O₂) at an anode. Due to the high heat of the chambers, water vapor also results from this process. If the hydrogen gas and oxygen gas were not effectively separated, such methods would result in an impure gaseous product that could not be effectively compressed or stored under pressure for industrial applications in a single container and is deemed explosive and dangerous. Thus, it remained desirable to develop a method by which a useful, stable, purified, compressible single gas could be formed from water.

### BRIEF DESCRIPTION OF THE DRAWINGS

FIG. 1 illustrates a schematic of a preferred reaction chamber for the invention.

FIG. 2 illustrates a graph (basolateral) showing the absorption of Vitamin C by cells treated with dioxytetrahydride gas-infused water and control.

FIG. 3 shows the effect on apical cells.

FIG. 4 illustrates the rate of gas absorption in water.

### DETAILED DESCRIPTION

In a first step of the method, water as an electrochemical solution is provided to a reaction zone to yield gas. The electrochemical solution is an electrolytic fluid comprising a salt dissolved in distilled water, where the salt is selected from the group consisting of potassium hydroxide, lithium hydroxide and sodium hydroxide and having a specific gravity greater than 1.0 and up to 1.3.

The electrochemical solution is provided to a reaction zone which is preferably closed off so to allow the reaction to occur under pressure. The specific gravity of the electrochemical solution is above 1.0 and not greater than 1.3.

The electrochemical solution is contained in a receptacle which can be made out of a variety of materials including sheet steel, stainless steel, CV-PVC and epoxy resin fiberglass. The apparatus and internal devices need to be heat resistant and waterproof.

The electrochemical solution is placed in a reaction zone in the method of the invention. Overall, the method employs creation of a magnetic field in which two oxygens are forced into a diatomic bond with four hydrogens. Under these conditions two water molecules bind in a stable gaseous state and there is no electrolysis or breaking of either the hydrogen or oxygen bonds to each other. Under these conditions, a single gas is generated and collected. This gas has desirable properties and is useful for various applications.

In a first step of the method, a magnetic field is generated in the reaction zone. Preferably, the magnetic field is applied by providing a source of electric power to said reaction zone. An electric current in said reaction zone provides a magnetic field.

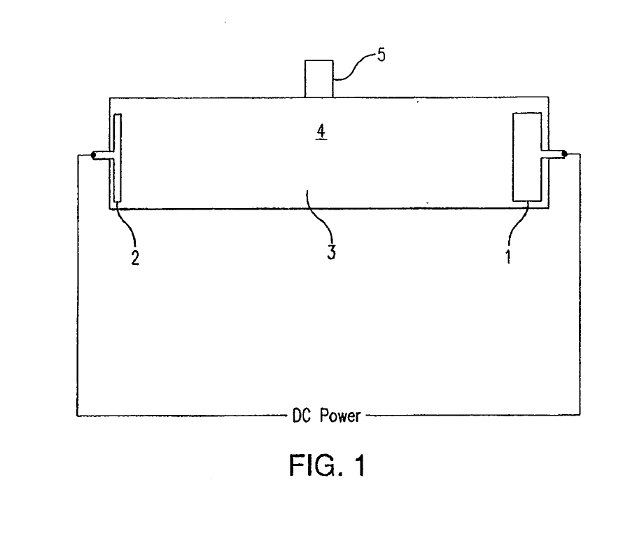

In a preferred embodiment, (see FIG. 1) two iron end plates 1 and 2 having an inside surface and an outside surface, and having the capacity to conduct an electrical current are used in the reaction zone 4 in opposing configuration. The inside of each end plate 1 and 2 is submerged in the electrochemical solution 3. The plates 1 and 2 should be separated a sufficient distance so that a magnetic field forms when current is applied to the reaction zone 4. The distance between the plates is approximately one-quarter inch.

There is a relationship between the concentration of electrochemical solution and the amperage which exists in the electrochemical solution upon application of current. The higher the specific gravity, the greater the amperage will be. This will also affect the strength of the magnetic field, and increase the temperature of the solution. Electrolysis (used industrially to produce hydrogen gas via the reaction 2H₂O(I)→2H₂(g)+O₂(g)) which is not desired in the method of this invention, could occur if the current is too high. The current may be too high if the specific gravity of the electrochemical solution exceeds the equivalent of 1.3 for potassium hydroxide.

In order for the magnetic field to be applied to the reaction zone, a power source (DC voltage) is applied respectively to the iron plates. This electromagnetic field inside the reaction zone transfers electrons through the electrochemical solution to associate with the four hydrogens of two water molecules with diatomic bond between the two oxygen, and the electrons repel other water molecules to create a stable gas (dioxytetrahydride).

Gas is generated not only at the iron plates but also appears as bubbles in the body of the electrochemical solution. A homogenous gas is produced in the body of electrochemical solution without the production of high levels of heat that would cause the water to vaporize (212° F.). Rather, the reaction zone remains at a temperature not exceeding 120° F. dependent on ambient temperature. Normally, there is a 30° F. temperature rise above ambient temperature assuming room temperature 90° F. In other words, conditions in the reaction zone are such that the temperature remains less than about 10° F. below phase transition temperature. The collection chambers contain no increase in oxygen gas, no increase in hydrogen gas, no hydrogen peroxide (a by-product of electrolysis) and no noticeable water vapor. Thus, costs are lowered, production speed increases, and the resulting gas is uniform in its properties. Also important is, the resulting homogeneous gas can be pumped into a stainless steel cylinder and has been found to be stable and not explosive under pressures of over 1000 psi.

The important functionalities in the process are imposition of a magnetic field on the electrochemical solution and flow of electrons through the electrochemical solution to generate the desired gas, under conditions short of those that will induce electrolysis. Over time in the operating reaction zone, only water is used to replenish the electrochemical solution and maintain the optimum specific gravity. In other words, only water is consumed in this process.

When water is converted into gas, the natural conversion from liquid to gas creates an increase in volume (1:1,800 increase) and thus an increase in pressure within the reaction zone. While standard atmospheric pressure is about 14.7 psi at sea level, the pressure in the closed reaction zone is maintained between one and 100 psi by using a pressure regulator at the outlet of the reaction chamber to control it, since maximum gas production occurs in this pressure range.

Now referring to FIG. 1, a schematic of a reaction chamber is illustrated. Iron plates (1) and (2) are in opposing configuration, approximately one-quarter inch apart. In the process of the invention, a current is passed through an electrochemical solution (3) and the current flow through the electrochemical solution creates a magnetic field. This produces the gas at a very efficient rate in the area of the electrochemical solution, as denoted by (4) in FIG. 1. The gas produced may be collected from the reaction zone through gas outlet (5) and subjected to further purification as taught herein.

The generated gas is then preferably exposed to a second magnetic field by providing a second reaction zone comprising rare earth magnets. The strength of the rare earth magnets should be greater than fifty (50) Gauss units. Gas flows through a chamber exposed to rare earth magnets for purification. Rare earth magnets, dense metal magnets typically made from a composite of neodymium, iron and boron with or without a nickel coating or plating, are attached to the exterior of the chamber. Since the gas is paramagnetic and water vapor is diamagnetic the magnetic chamber strengthens the molecular bond of the gas and repels the water vapor back into the either the electrochemical solution or is captured in a dryer.

The purified gas may be used immediately or compressed and stored in a gas storage tank. Purified gas may be allowed to flow out of said second reaction zone directly to a torch attachment, to a compressor for storage in a pressurized vessel, or gas outflow valve for infusion into water or other substances.

In a method for making a compressible, stable gas with desirable properties, gas is made according to the method of the invention. The dioxytetrahydride gas can then be safely compressed and stored. The gas can be compressed above 1,000 psi. The gas also can be stored in a pressurized vessel.

In an exemplary procedure for compression, the gas is discharged from the apparatus into a hose with a compressor attached. A Whirlwind Compressor, Model 2200-2 HPE, manufactured by High Pressure Eng. Co., Inc. (an oil-less compressor) can be used. A canister with pressure gauges is used to fill the chamber with the gas, using a hose to transport the gas from the apparatus and compressor into the canister. An empty oxygen tank that has been vacuumed to remove any residual oxygen and water, is used. The empty and vacuumed oxygen tank with a pressure valve has a manufacturer name of White Martins, ABRE with dimensions of 23″ diameter and 19″ height. The gas is placed under pressure in the compression chamber up to and beyond 1,000 psi. for storage of the dioxytetrahydride.

The gas remains stable and under pressure for one month and longer. To test its stability, wood chips were placed in a stainless steel tank and the tank filled with the gas. The wood chips absorbed the gas and additional gas was used to refill the chamber and maintain a pressure of 30 psi. Once the wood chips were saturated with the gas, the tank was decompressed and pressure reduced to 0 psi. For a period of over 30 days, no pressure was generated showing that no out-gassing of the gas occurred. The wood chips displayed different burn properties after 60 days when compared to that of the non-treated wood chips. The treated wood chips with absorbed gas burned more efficiently when compared to that of non-treated wood chips thereby demonstrating the stability of the gas bond with the treated wood chips.

### Analytical Testing and Observations of Dioxytetrahydride Under Pressure

Maximum Pressure: The gas does not implode even when pressures exceed 1,600 psi. Instead, the gas requires a reaction such as a spark generated by a fitting failure of a high-pressurized chamber or exposure to a flame to cause the gas to implode.

Safe Pressurization: The gas remains safe and stable at pressures around 1,000 psi for over 30 days. The gas should remain stable under pressure indefinitely, at least for a sufficient period of time to allow the gas to be utilized 30-60 days after generation.

The purified gas was tested and exhibited properties of a pure, homogeneous gas that was found to be compressible as stated above, safe, and able to oxidize any non-oxidized substrate its flame contacted and, it was able to reduce any completely oxidized substrate its flame contacted. The following characteristics were observed.

Ultra-violet Light Test: Exhibits a blue gray color appearance compared to untreated distilled water which exhibits no color, when exposed to an ultra-violet light manufactured by Zelco Industries Model 10015.

Balloon: Is lighter than air and causes balloons filled therewith to rise.

Cooling: The Balloon Filled with purified gas: Balloon remains inflated at or below −10° F.

Ignition: The purified gas produced according to the above method was tested for ignition properties. The purified gas, when lit with an ignition source such as a spark, causes an implosion. The temperature of the flame produced upon ignition was estimated to be about 270° F. using an infrared temperature device (Raynger ST2L infrared temperature device). However, when materials are exposed to the flame, which creates a chemical reaction with the material, base metals will rapidly rise to melt temperature points, releasing heat and converting the gas back into water (H₂O).

Purified gas was discharged from the reaction zone through a hose with a torch attached. On the gas output of the apparatus, a flash-back arrestor is recommended. The gas may be exposed to an ignition source (e.g., spark or electrical arc) and combustion of the gas occurs. The heat of the resulting flame on the subject torch has a temperature of approximately 270° F.

When an air/propane torch is burning, a small amount of gas is introduced into the air mixing chamber, a single uniform flame cone becomes visible demonstrating a more efficient conversion of hydrocarbon and more heat from combustion of hydrocarbon, meaning it has a use as a fuel extender. One use is injection of gas into an air intake of a combustion engine thereby reducing harmful exhaust emissions and increasing fuel efficiency. A by-product of this process is the creation of water during the combustion cycle that generates steam. The steam causes an increase in the torque generated by the engine resulting in greater power output. The steam causes an increase in the torque generated by the engine resulting in greater power output. The gas extends fuel efficiency. When ignited purified gas contacts zo another substance, melting occurs within a short period of time, usually less than one minute. The results of some examples of substances exposed to ignited purified gas may be found in Table 1.

TABLE 1
Effect of Ignited dioxytetrahydride on Various Substances

| | | | Effect on Exposure to |
|---|---|---|---|
| | | | Ignited Purified Gas |
| Substance | Melting Point | | (one minute or less). |
| Stainless Steel | 2,600° | F. | Melting. |
| Steel | 1,330° | F. | Melting. |
| Copper | 1,984° | F. | Melting. |
| Ceramic | 10,000° and 12,000° | F. | Melting. |
| Tar Sands | | | Sand converted to |
| | | | glass and metals were |
| | | | separated out of the |
| | | | sand matrix. |
| Concrete | | | Creates a glassy |
| | | | molten surface which |
| | | | can adhere to metal |
| | | | when cooled. |
| Glass | | | Melts. Flame and |
| | | | true colors are |
| | | | achieved with no |
| | | | carbon flakes or |
| | | | residue embedded |
| | | | inside the glass. |

In lieu of melting a substrate, ignited purified gas may be applied to a substrate with a view toward capturing the generated heat as a useful product. The heat generated can be transferred to a substance such as air or water, thereby producing hot air or steam that can then be used industrially, such as for example to drive a turbine or piston-type engine for production of mechanical energy. In a preferred method, the flame of the gas can be applied to a substrate in conduit form having an inside surface and an outside surface. A substance such as forced air or water can flow thorough the conduit adjacent the inside surface of the conduit. The flame of the gas can io be applied to the outside surface of the conduit which causes the heat-generating reaction to occur. The heat is then transferred to the substance flowing through the conduit, preventing melting of the surface but creating a useful heated fluid that can be used in further applications. An exemplary conduit is a metal tube or pipe, such as copper tubing. It has been further determined that gas can be infused into other substances, rendering a useful product.

Candles: Gas infused into melted paraffin wax and poured into a mold with a wick will create candles that burn with lower carbon emission as observed using a Pace 400 Four Gas Analyzer.

Fluids: The gas has an affinity for water and other liquids including fuels but they bubbled from the liquids after reaching a saturation point. One novel use of the gas is infusing it back into water to create ionized or polarized water. The resulting gas-infused water creates smaller water clusters that are believed to permit faster cellular absorption and hydration.

In an exemplary method for infusing gas into water, gas is discharged from the reaction zone into a hose with a ceramic diffuser attached. For treating large volumes of water, a ceramic block diffuser may be used. The diffusers are used to reduce the size of the gas bubbles to improve efficiency of water absorption. Gas may also be stored under pressure, and then infused into water.

It is preferred to infuse water that has gone through a distillation process prior to infusion of gas into treated water with less than 1 ppm TDS (Total Dissolved Solids). One may use an absorption graph to determine time required for achieving desired absorption of gas into water. The typical rate of 30% absorption is approximately one hour to treat 100 gallons of water. A higher saturation of gas up to 100% of total absorption occurs with more infusion of gas into water over time. The actual time and percentage of absorption of gas are affected by the purity of water, volume of water, size of gas bubbles, temperature and other factors.

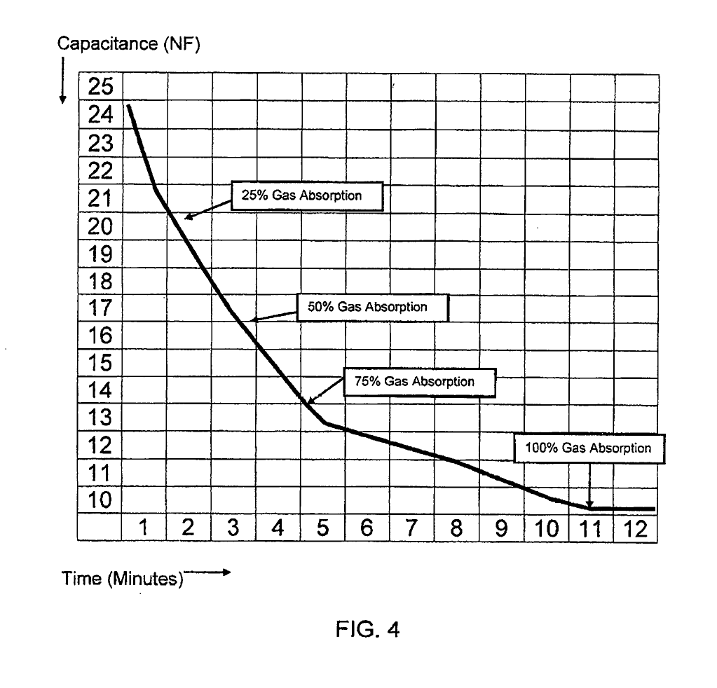

The resulting ionized or polarized water (“Gas-infused Water”) clings longer to a magnet when compared to that of regular water. Absorption over time or saturation graphs to monitor changes in the water properties infused with Gas including capacitance levels may be prepared. FIG. 4 shows a typical absorption over time graph for infusion of the gas into water. Subsequently, one may measure capacitance levels in the treated water over a time period exceeding 30 days to demonstrate that the gas in water is stable.

Other measurement: Total Dissolved Solids (TDS) dropped from a start of 0.33 ppm in untreated distilled water to a finish of 0.17 ppm after infusion of the gas into distilled water for a period of approximately 11 minutes. A Fluke 189 True RMS Multimeter was used to measure drop in capacitance.

Storage of Gas in Water: The resulting polarized water with Gas treatment remains stable and can be stored for 2 years or more. The actual maximum storage time has yet to be observed but in theory, Gas should remain permanently stable in the water.

Absorption: During infusion of Gas into purified water, we used a Fluke 189 True RMS Multimeter to measure drop in capacitance. The absorption over time graph is plotted to monitor the drop in capacitance. The first capacitance drop during initial infusion of the gas into a gallon of purified water occurs within the first three minutes of infusion. After that time, the capacitance gradually drops until the point of maximum saturation of the gas is typically reached between eight and 20 minutes depending on variables including initial purity of water, size of gas bubbles, and volume of water to be treated. The resulting treated or infused water is referred herein as gas-infused Water.

Other Parameters Monitored: During infusion of the gas into purified water, a drop in TDS (Total Dissolved Solids) concentration, conductivity and resistively can be measured. An appropriate measuring device is a Control Company Traceable™ #4063CC meter.

pH Test: Lab tests show that distilled water had a pH of 6.8 and when infused with the gas had a pH change to 7.6.

Ice Cubes: Gas remains in gas-infused Water or polarized water until freezing temperatures when the gas forms a gas bubble within the ice cube itself, sometimes producing on the surface of the ice cubes, capillary tubes where the gas escapes.

Ultraviolet Light Exposure: Gas-infused water was tested for the effects of ultraviolet light exposure. A clear spray bottle containing gas-infused water or polarized water placed in the Florida sun for over two years remained clear in appearance and without algae growth which had been observed in water not infused with the gas under similar conditions.

Magnets: A drop of gas-infused water clings to the surface of a magnet longer when compared to that of untreated water.

Many uses have been found for gas-infused Water. Table 2 lists some of those uses.

TABLE 2
USES FOR DIOXYTETRAHYDRIDE GAS INFUSED WATER

| | Advantages Provided Over |
|---|---|
| Use | Untreated Water |
| Drinking water for human and | Efficient cellular absorption and |
| animal consumption and hydration | removal of toxins. |
| Water for food and health | Pure form of water that improves |
| supplement manufacturing, | product quality, shelf life, nutrient |
| preparation, and cooking | benefits, absorption, and taste. |
| Water for cleaning and enhancing | Reduced need for emulsifiers and |
| effectiveness of cleansers | surfactants. |
| Water for plants and crops | Greater size of plants, improved plant |
| including hydroponics, floral | quality, longer viability, and reduced |
| arrangements and turf | scale buildup including in hydroponic |
| (golf courses) | water containers. |
| Fertilizer solution for application | Higher yield and more vigorous |
| on plants and crops | growth. |
| Water for aquariums and fish | Greater size of fish. |
| farming | |
| Water systems including long-term | Less algae growth resulting from |
| water storage, municipal supplies | antibacterial properties. |
| and in-home treatment systems | |
| Steam, air heating and air | Less algae or mold growth for cleaner |
| conditioning systems | air circulation systems. |
| Refrigeration systems | Less mold accumulation. |
| Industrial scrubbers | Less algae growth and scale buildup |
| | to maintain scrubbing efficiency. |
| Industrial products and processes | Reduce or eliminate need to use |
| including oil, gas and tar sand | petroleum-based solvents. |
| extraction | |
| Pharmaceutical and medicine | Efficient carrier of medicines and |
| manufacturing | removal of by-products from |
| | medicines and solvent carriers. |
| Skin treatment products | Hydration of skin cells, improved |
| | absorption of moisturizers, and |
| | reduction in pigment changes due to |
| | sun damage. |
| Wound treatment products | Faster healing and pain relief. |
| Respiratory relief used in | Improved breathing with less snoring. |
| humidifier systems | |
| Eye relief products | Relief for irritated eyes and |
| | hydration. |
| Dental care products | Removal or inhibit plaque and stains |
| | on teeth. |
| Cosmetics and beauty supplies | Less need for chemical binders and |
| | more resistant to contamination |
| | buildup in cosmetics; improved hair |
| | growth. |
| Water features including | Cleaner water with less or no chlorine |
| swimming pools, spas, hot | and chemical additives. |
| tubs, waterfalls, fountains, | |
| water amusement parks | |

The gas herein is disclosed to be an ionized H₄O₂ gas with the potential to oxidize or reduce any substance. On a non-oxidized substrate, such as steel, the active oxygen within the molecule will chemically bond to the steel bringing it immediately to its melting temperature and releasing hydrogen, which bonds with atmospheric oxygen to produce heat. On an oxidized substrate, such as ceramic, the hydrogen reduces the substrate by chemically bonding with the oxygen present within the substrate, melting the material and io releasing atomic oxygen, which then bonds with the material. This double reaction is responsible for producing much more heat than an ordinary oxidation reduction reaction.

These reactions are proven on rusty steel and concrete. When ordinary gas, such as: methane, ethane, propane, butane, or acetylene are applied to rusty steel, popping and spitting of material occurs due to the explosive reaction of the ferrous oxide being separated from the non-oxidized metal due to different expansion rates.

With the inventive gas, this does not occur, since oxidation and reduction are occurring at the same time and the expansion rates are equal. On concrete when heat from an ordinary gas is applied, the portion the flame touches will expand and break loose from the rest of the concrete with an explosive force and spit pieces of hot concrete outward and leave holes in the concrete surface. Again, this does not occur with the inventive gas because it is being reduced to a liquid form before the pressure of uneven expansion occurs.

Simply stated the inventive gas herein is an ionized H₄O₂ gas capable of oxidizing or reducing almost any material without the adverse reactions created by heat producing flames. Heat is the byproduct of friction, in chemistry two atoms colliding together in a reaction known as oxidation and reduction cause this friction. A gas, referred to as a fuel, is usually a hydrocarbon that is easily oxidized, however, the carbon is what is being oxidized and the oxygen is being reduced meaning this is where friction occurs and these are the items being heated. Heat given off by these substances is refractive heat and the substances being heated are absorbing heat or, better stated, are being bombarded by fast moving hot gases. Gas may change the definition of melting point due to the lack of heat producing flames.

Use in Process of Tar Sands Extraction: Conventional water with petroleum solvents used in the separation of tar from sand was replaced with gas-infused water. Gas-infused water was heated (no petroleum solvent added) with a sample of tar sands in a pan to approximately 160° F. Tar was observed separating from the sand, providing a cleaner and more efficient process with less by-products and emissions released from tar extraction.

Use for Improved Cleaning: For laundry, one may add a quantity (⅓ of a gallon in a standard washing machine tub of 12 gallons for medium load and 16 gallons for large load) of gas-infused water to the soap cycle of a top loading washing machine and the remaining water (approximately ⅔ of a gallon) is added to the rinse cycle. The polarized characteristic and smaller molecular size of gas-infused water enables the detergent and water solution to more thoroughly penetrate the cloth fabric and remove the dirt and grime. The addition of gas-infused water to the rinse assists in completely removing the soap residue that may contain residual dirt from the fabric. This process results in cleaner and stain-free laundry with less body oil and bacteria buildup. Laundry without these gas-infused water additives display less brilliant whites and retain a pungent odor caused by residual bacteria living in the fabric of the washed clothes.

Reduced Use of Emulsifiers and Surfactants: One may dilute cleaning solutions with gas-infused water for effective cleaning of surfaces to remove grime, oil and grease and removal of bacteria. Gas-infused water is a natural disinfectant without harsh chemical additives. Typically, one uses at least 1 part cleaning solution with 20 parts gas-infused water to maintain cleaning properties.

### Biological Properties

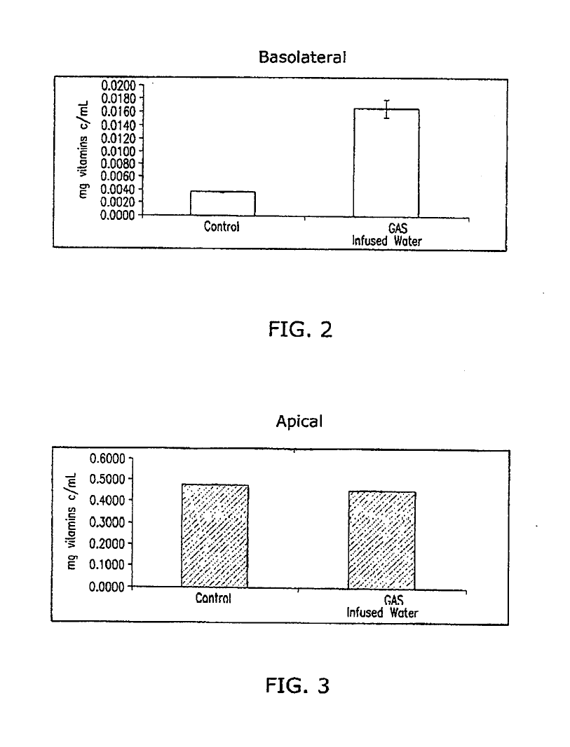

Transport, Delivery and Absorption of Nutrients: In a controlled experiment, a standard drug metabolism test in vitro was conducted over a period of 21 days. This comparative test was performed on cell membrane permeability for Vitamin C solution (L-ascorbic acid) using (1) Hank's Buffered Saline Solution (HBSS) and (2) gas-infused water. Caco-2 cells were used and permeability of the apical side (similar to intestine surface) and basolateral side (similar to underneath intestinal surface) for the separate solutions were determined. Vitamin C quantification was conducted on HPLC (HP1100 equipped with PDA detector) and Zorbax C18 reverse phase column (4.6×250 mm, 5 micro) at 30C. Test results demonstrated Vitamin C permeability of gas-infused water was about 4 times higher than the control counterpart. (Hu, 2008 (unpublished communication). Results are provided in FIGS. 2 and 3.

Plant Growth: In a controlled greenhouse setting, four groups of ivy plants were watered using (1) 100% well water, (2) mix of ⅓ mix gas-infused water and ⅔ well water, (3) mix of ⅔ gas-infused water and ⅓ well water, and (4) 100% gas-infused water. The ivy plants were harvested and dehydrated to allow measurement of dry plant mass. The fourth group of 100% gas-infused water had over 16 percent increase in mass when compared to that the first group of well water. (Reiser, 2006 (private communication).

Fish Growth: Two home aquariums were used to hold two respective groups of goldfish. Gas was bubbled into one aquarium and the second with air for a period of thirty days. It was observed that the goldfish in the former aquarium aerated by gas grew at least 15 percent more and the aquarium tank remained cleaner with less algae growth.

Wound Treatment and Healing: The polarization of the gas-infused water provides natural anti-bacterial and non-toxic anti-infective properties that promote healing of superficial and multi-layer wounds and a reduction in pain perception. A fifty-year old woman burned herself by accidentally spilling scalding-hot coffee onto her hand. Upon seeking medical attention, a zo physician advised the patient that she may have to undergo abridement or dead skin removal and possible skin graft surgery. The patient washed the affected area with gas-infused, purified water and applied a medicinal ointment. The wound was wrapped with a sterile gauze and the gauze was moistened to keep the wound hydrated with gas-infused water. The patient reported an immediate and on-going lessening of pain with the application of gas-infused water. Over the period of ten days with repeating these treatment steps involving changing of the moistened sterile gauze on at least a daily basis, the site of the wound developed new skin with minimal evidence of scarring.

Upon cessation of the treatment regime when the upper skin layer appeared to be healed, blisters appeared on the surface of the skin. The treatment with gas-infused water was reinitiated and the blisters healed as well as the remaining layers of skin. The patient experienced healing and thereby avoided debridement of dead skin, and skin grafts.

Skin Treatment: Topical applications twice a day on each side of a male volunteer's face in vicinity of his eyes were made. Two types of topical solutions were prepared with 1% magnesium ascorbyl phosphate (MAP), one using gas-infused water and the other using tap water. After 21 days, the volunteer observed on the side where gas-infused water solution was applied, a slight reduction in the depth of fine lines around the eye and a lighting of darker skin pigment when compared to that of the other area where the tap water solution was applied. (Puleo of Optima Specialty Chemical, 2008 (private communication).

Eye Relief: Gas-infused water may be sprayed into the eyes for immediate relief and lessening of redness that is comparable to use of over-the-counter eye drops. This natural treatment without any chemical additives, assists in hydrating eyes and removing irritants such as dust and pollen.

Dental Care: A 50:50 solution of commercial mouth wash was mixed with gas-infused water and a capful of this solution was used twice a day after brushing teeth. Less plaque buildup and stains were noted by professional dental hygienists as compared to previous observations six months earlier when this solution had not been used.

### Molecular Structure Based on Gas Properties

It is believed by the inventors from observing the properties of the gas that the process disclosed herein results in a product not achieved by heretofore-reported processes for the conversion of water into gas.

Given the low energy reaction that created the gas and the use of no catalysts, it is believed unlikely that any O—H bonds of water could possibly be broken in the process used. It is known that breaking O—H bonds requires two faradays per mole and the process of the invention only employs 2.8 watt hours per liter, which is about a maximum of 1.6 faradays per mole. Further, the Gas resulting from the process disclosed herein is flammable but the flame temperature of the gas is only about 270° F. (132.2° C.), as compared to diatomic hydrogen gas which is highly combustible and auto ignites at 560° C. A hydrogen/oxygen torch flame is reportedly 3200° C.=5792° F. However, the gas flame easily melts metals, which likely indicates that an oxygen is active. The gas flame also reduces ceramics, which indicates that the hydrogen is in an ionized state.

The gas has an affinity for water and other liquids including fuels, but bubbles from the liquids after reaching a saturation point. One use of the gas disclosed herein is infusing it back into water to create ionized or polarized water.

The gas is always a gas at room temperature while normal water vapor requires energy to evaporate in great quantities. When com busted, the gas always returns to liquid water. When placed in a balloon, the gas initially floats the balloon but it seeps from the balloon rather quickly indicating that the gas has a small molecular structure.

Gas is an electron-rich gaseous form of water not created by electrolysis, but rather by creating an electron rich environment with a targeted, energy efficient process that provides a reaction zone for gas to form. Gas is created when very strong, targeted electromagnetic fields are applied to liquid water in the presence of an electrochemical solution. The electromagnetic energy provided is sufficient to promote formation of diatomic oxygen from two water molecules. The arrangement of the remaining hydrogen from the same water molecules is also impacted by the process. These same excess electrons provide the targeted energy to overcome the normal H—O—H water geometry and allow a larger, yet stable arrangement of hydrogen and oxygen to form, (ie: H₄O₂). The same electron rich environment promotes hydride character of the hydrogen portion of this new structure and allows its bond angle to be altered between the hydrogen and oxygen, (from 104° to 118°). In addition, the same hydride character of hydrogen portion of this new arrangement provides the stability for its new geometry, inhibits “proton like” clustering, and helps maintain a stable, autonomous, independent H₄O₂ liquid-gas structure.

In generating Gas, there are no appreciable hydrogen Cations (H⁺) or Anions (H−) formed in the completed reaction. However, the provision of an electron rich environment promotes the hydride behavior in the hydrogen io portion of the (H₄O₂) structure. It is this hydride effect that prevents conventional ionization, and inhibits the gas molecules from clustering like the H⁺ proton. In other words, the electrons repel other available water molecules and this in turn, allows enough stability for the (H₄O₂) to form, (not cluster), and exist in an autonomous, stable, water-liquid gas, (H₄O₂), independent molecular structure. It is the addition and surplus of electrons that repel water molecules to create the gas of this invention and not the breaking of either hydrogen or oxygen bonds like that which occurs in classic electrolytic wet chemistry. This absence of bond breaking explains how such a low amount of energy is used to convert liquid water into gas with this process.

The inventors have discovered of a new isomer of water. The oxygen is active and can oxidize metals. In the unburned gaseous state, the increased negative charge causes greater spacing among the gas molecules causing stability, a lower boiling point, a lower freezing point, and a higher vapor pressure. The gas does not cluster to create liquid water at regular atmospheric temperatures and pressures as does the molecules of normal water vapor. The gas exists in a higher energy state, burns by itself at a low temperature, and melts any substrates when exposed to the gas flame. The gas flame has a uniform blue color appearance without yellow sparks indicative of water (H₂O) vapor, or red sparks, indicative of either H₂ or O₂ gas contamination.

### Gas Discharge Visualization (GDV) and the Kirlian Effect

Currently, the term Kirlian effect is used to describe the visual observation and digital photographic capturing of a biological subject. A glow or “energy field” surrounds the object's surface when it is placed in a charged electrical field and photographed using a gas discharge emission. The results of capturing these biological subjects is known as “bioelectrography” or electrophotonics, as well as Kirlian photography. The analysis of infused water from the inventive process was undertaken by Dr. Zachary Bush, M.D. with peer review by Mary Milroy-CEO, GDV Source, BSIE, Dipl. Esogetic Medicine, Dipl. Ac & CH (NCCAOM).

In using a GVD (Gas Discharge Visualization) Camera, droplets of many types of water, including the gas-infused water of this invention, were suspended and energy measurements were taken. One type of water tested was water infused with Brown's gas or gas produced from electrolysis (named “Sparks”). The test results for the gas-infused water were significantly novel from those of other types of water tested, with a sixteen standard deviation difference of the gas-infused water from the other types of water tested including “Sparks” water. Again using the GVD camera and the same testing protocol, gas-infused water produced over seven years before this testing had similar results. These results of the gas-infused water show a very high level of energetic activity and stability.

One of the theories on the gas-infused water with its H₄O₂ (double water molecule) structure enriched with electrons is that it is more compatible with that of humans, animals and plants when compared with a H—O—H (single water molecule) structure. Electrons associated with hydrogen are used in the Krebs Cycle to allow cellular mitochondria to produce energy for the body. The remaining oxygen is more benevolent to the body if left in a diatomic (O₂) state as opposed to a single nascent oxygen (O) which can contribute to oxidative stress that in turn, can lead to malignant tumors and other diseases of the body.

In the process used herein, electrolysis does not take place. “Electrolysis” is defined as a “method of separating chemically bonded elements and compounds by passing an electric current through them.” Electrolysis does not take place and no splitting of the water molecular bonds occurs, as is demonstrated by the fact that no increase in hydrogen or oxygen gas can be measured in the reaction zone. This is a key differentiator from the processes that have resulted in a gas being produced by electrolysis of water. The gases produced by electrolysis exhibit far different properties from the gas discovered herein. Gases produced by electrolysis are explosive, cannot be pressurized and are heat-producing gases on ignition. One method to prove no electrolysis is to test Oxidation Reduction Potential (ORP) of pure water infused or saturated with gas in it. ORP was tested by a nationally-recognized lab before and after “sparging” with nitrogen gas. Duplicate nitrogen sparge tests were performed on duplicate samples from the same aliquot of above said inventive gas saturated solution. Before ORP's were measured at 379 mV (millivolts) and 380 mV respectively. After ten minute continuous sparge with nitrogen gas, the ORPs were measured at 345 mV and 342 mV respectively. The 9 to 10% drop in ORP was explained from the effect of the plastic sparger tube surface immersed in and connected to the zo fish tank stone sparger consuming some of the ORP. The conclusion: if Gas was ionized hydrogen and/or oxygen (by-products of electrolysis), then the amounts of these ions would have been measured much higher in solution before sparging, and the ORP would have changed notably as soon as the nitrogen gas was allowed to sparge into the inventive gas saturated solution.

What we claim is:

1. A method for making a gas comprising the steps of:
(a) providing a volume of water in an electrochemical solution to a reaction zone comprising a receptacle and a gas output means, wherein said reaction zone is closed and may withstand pressure of at least 30 psi and wherein said electrochemical solution has a specific gravity below the equivalent of 1.3 for potassium hydroxide;
(b) providing a magnetic field to said reaction zone under conditions which will not induce electrolysis of said water; and
(c) collecting said generated gas.

2. The method of claim 1, wherein said magnetic field is provided by means of two opposing iron plates spaced approximately one-quarter inch apart.

3. The method of claim 1, further comprising the step of compressing the generated homogenous gas and storing in a pressurized container.

4. The method of claim 3, wherein said generated homogenous gas is compressed and stored in excess of 1,600 psi.

5. The method of claim 4, wherein said generated homogenous gas is pressurized to over 1,000 psi.

6. The method of claim 5, wherein said generated homogenous gas is storable for at least 30 days.

7. The method of claim 1, wherein the conditions in the reaction zone are such that the temperature remains less than about 10° F. below phase transition temperature.

8. The method of claim 1, wherein the conditions in the reaction zone are such that the pressure does not exceed 100 psi.

9. The method of claim 1, wherein the generated homogenous gas is exposed to a second magnetic field by providing a second reaction zone comprising rare earth magnets.

10. The method of claim 9, wherein said rare earth magnets have a strength greater than fifty (50) Gauss units.

11. The method of claim 10, wherein said generated gas flows through said second reaction zone comprising said rare earth magnets.

12. The method of claim 11, wherein said rare earth magnets comprise a composite of neodymium, iron and boron.

13. The method of claim 12, wherein said rare earth magnets further comprise a nickel coating or plating.

14. A generated gas product from the method of claim 1, which is an electron-rich, autonomous, independent, molecule which is dioxytetrahydride having the formula H402 .

15. A generated gas product as claimed in claim 14 which has a pressure of at least 1000 psi and which is odorless, colorless under visible light, blue gray under an ultraviolet light, and is flammable when exposed to an igniting source.

16. The generated gas product as claimed in claim 14 characterized by producing a very low temperature flame of approximately two hundred seventy degrees Fahrenheit upon being ignited.

17. A method of melting materials by igniting the gas as claimed in claim 14 in a controlled manner and putting the resulting flame in contact with said materials.

18. The method of claim 17 wherein said gas is characterized when burning in contact with a substance, causing a chemical reaction such that said substance rises to an intrinsic melting or oxidation point.

19. The method of claim 18, wherein said substance is selected from the group consisting of stainless steel having an intrinsic oxidation point of 2,600° F., steel having an intrinsic oxidation point of 1,330° F., or copper having an intrinsic oxidation point of 1,984° F., and which melt when exposed to the said gas flame for less than one minute.

20. The method of claim 19 wherein said substance is selected from the group consisting of ceramics or silicon dioxide.

21. A method for generating heat useful for transfer to a fluid selected from the group consisting of air or water, thereby producing hot fluid for production of mechanical energy, comprising contacting a substrate with a dioxytetrahydride flame under conditions that permit transfer of heat from a reaction of said dioxytetrahydride with said substrate, to said fluid.

22. The method of claim 21, wherein said substrate comprises a conduit through which said fluid is flowing at the time of contact with said flame, and whereby said fluid is heated and flows away from a reaction site to a site where it is used to generate mechanical energy or heat.

23. A method for infusing dioxytetrahydride into an air intake of a combustion engine thereby producing and extending the energy of a fuel used for mechanical energy, comprising contacting dioxytetrahydride with said fuel under conditions that improve chemical reaction of said fuel during its combustion cycle through generating more energy from said fuel.

24. The method of claim 23, wherein said combustion engine comprises a conduit through which said fuel is flowing at the time of contact with said dioxytetrahydride, and whereby said fuel combusts at a reaction site where said fuel more efficiently combusts to generate mechanical energy and less harmful emissions.

25. Water infused with dioxytetrahydride.

26. A method for using water infused with dioxytetrahydride for ingestion, said process comprising ingesting at least 1 ounce of said water.

27. A method for using water infused with dioxytetrahydride for providing improved growth to biological systems, said process comprising treating said biological systems periodically with said infused water.

28. A method for using water infused with dioxytetrahydride for improving uptake of nutrients by animal cells, said process comprising periodically treating an animal with said infused water, thereby treating said animal cells.

29. The method of claim 28, wherein said nutrient is at least one vitamin.

30. The method of claim 29, wherein said vitamin is Vitamin C.

31. A method for using water infused with dioxytetrahydride for improving uptake of fertilizers by plant cells, said method comprising periodically treating said plant cells with said water infused with dioxytetrahydride.

32. A method of treating skin with water infused with dioxytetrahydride, said method comprising treating said skin periodically with water infused with dioxytetrahydride.

33. The method of claim 32, wherein said skin is dehydrated and damaged.

34. A formulated solution for eye treatment, said formulated solution containing at least dioxytetrahydride infused water.

35. A method for reducing the amount of surfactant or emulsifiers in a cleaning formulation, said method comprising adding a predetermined portion of dioxytetrahydride infused Water to said cleaning formulation.

36. A method for cleaning, said method comprising applying a formulation containing at least dioxytetrahydride infused water to a surface.

37. The method of claim 36, wherein said surface comprises teeth.

38. The method of claim 36, further comprising adding water infused with dioxytetrahydride to a mouthwash and contacting said surface with said mouthwash.

39. A product which is water, infused with dioxytetrahydride, wherein said dioxytetrahydride is produced by a method comprising the steps of:
(a) providing a volume of water in an electrochemical solution to a reaction zone comprising a receptacle and a gas output means, wherein said reaction zone is closed and may withstand pressure of at least 30 psi;
(b) providing electricity to said reaction zone under conditions which will not induce electrolysis of said water, but will provide a magnetic field, wherein the quantity of electricity transferred to the reaction zone does not exceed two faradays/mole;
(c) collecting said generated dioxytetrahydride.
